(19) United States
(12) Patent Application Publication
(10) Pub. No.: US 2020/0385462 A1
(43) Pub. Date: Dec. 10, 2020

(54) ANTIBODY FOR USE IN DETECTING EARLY-STAGE DAMAGE OF BLOOD BRAIN BARRIER DURING CEREBRAL ISCHEMIC STROKE AND APPLICATION THEREOF

(71) Applicant: XUANWU HOSPITAL OF CAPITAL MEDICAL UNIVERSITY, Beijing (CN)

(72) Inventors: Zhifeng QI, Beijing (CN); KeJian LIU, Beijing (CN); Xunming JI, Beijing (CN)

(73) Assignee: XUANWU HOSPITAL OF CAPITAL MEDICAL UNIVERSITY, Beijing (CN)

(21) Appl. No.: 16/910,058

(22) Filed: Jun. 23, 2020

Related U.S. Application Data
(63) Continuation of application No. PCT/CN2018/121503, filed on Dec. 17, 2018.

(30) Foreign Application Priority Data
Dec. 25, 2017 (CN) 201711424114.9

(51) Int. Cl. C07K 16/28; G01N 33/68; G01N 33/543

(52) U.S. Cl. CPC C07K 16/28; G01N 33/6896; G01N 2800/2871; C07K 2317/34; G01N 2333/705; G01N 33/54306

(57) ABSTRACT

A specific antibody for detecting the blood brain barrier early injury of cerebral ischemic stroke is characterized by specifically identifying specifically identifying DHYETDYTTGGESC in degradation fragments of an occludin protein, but not identifying the full-length occludin protein. Therefore, the antibody can be used for specifically detecting the blood brain barrier early injury of the cerebral ischemic stroke, and can eliminate the influence of the full-length occludin protein in serum on a detection result, so that the specificity and accuracy of detecting the blood brain barrier early injury of the cerebral ischemic stroke are significantly improved.

### CROSS-REFERENCE TO RELATED APPLICATIONS

The present application is a continuation of, and claims priority to, PCT/CN2018/121503 filed on Dec. 17, 2018, which claims priority to Chinese Patent Application No. CN 201711424114.9 filed on Dec. 25, 2017. The disclosures of these applications are hereby incorporated by reference in their entirety.

### SEQUENCE LISTING

The instant application contains a Sequence Listing which has been submitted electronically in ASCII format and is hereby incorporated by reference in its entirety. Said ASCII file, created on Dec. 25, 2017, is named KP1850688-O-Sequence-Listing-Corrected.txt and is 4,739 bytes in size.

### BACKGROUND

Stroke, due to its high incidence and high disability rate, has become a major disease that seriously threatens human health at present, 80% of which is cerebral ischemic stroke. The latest “Global Burden of Disease Study” published in The Lancet shows that cerebral stroke has become the first cause of death in China. Due to the accelerated aging of the population and poor control of risk factors, the incidence of cerebral stroke in China is rapidly rising at an annual rate of 8.7%. The annual treatment cost for stroke patients in Beijing alone exceeds 10 billion yuan, which is a heavy social and economic burden.

### SUMMARY

The present disclosure relates generally to the field of disease detection, and more specifically to a specific antibody for detecting the blood brain barrier early injury of cerebral ischemic stroke, and use thereof.

An objective of the present disclosure is to provide an antibody capable of specifically identifying degradation fragments of occludin in serum, and use thereof. By detecting a patient with the antibody, the influence of the full-length occludin protein in serum on the detection results can be eliminated, and the specificity of the detection result is obviously improved. By detecting the concentration of protein fragments in a sample, it can be used for judging whether cerebral ischemic stroke occurs and the degree of onset.

In a first aspect, provided is an antibody capable of specifically identifying degradation fragments of occludin in serum, and the antibody can be used for specifically detecting blood brain barrier early injury of cerebral ischemic stroke.

The antibody is characterized by only specifically identifying DHYETDYTTGGESC in the degradation fragments of the occludin protein, but not identifying the full-length occludin protein.

Further, a preparation method for the antibody includes the following steps:

(1) synthesizing a polypeptide DHYETDYTTGGESC;

(2) synthesizing an antigen: coupling the polypeptide synthesized in the step (1) with KLH to obtain KLH-polypeptide as an immune antigen;

wherein, during coupling, SuLfo-SMCC is selected as a coupling agent;

(3) immunizing animals with the immune antigen to obtain antiserum; and

(4) performing antibody purification on the antiserum with an antigen affinity column to obtain a specific antibody.

It should be noted that, the above polypeptide sequence represents a sequence of a female parent protein. A chemically-synthesized polypeptide often carries a free amino group and a free carboxyl group, and thus in order to be more similar to the female parent protein, the ends of the polypeptide often need to be blocked, i.e. N-terminal acetylation and C-terminal amidation. These modifications will reduce the total charge of the polypeptide, reduce the solubility of the polypeptide, and also enable the polypeptide to simulate its original state of a amino groups and carboxyl groups in the female parent protein.

The antigen affinity column is prepared by connecting the polypeptide synthesized in the step (1) to an activated SuLfolink Resin.

The animals can be rabbits, mice, sheep, horses, etc. In a specific embodiment of the present disclosure, New Zealand white rabbits are selected as immune animals, but the immune animals that can be used in the actual preparation of the antibody are not limited to this.

On the basis of using the New Zealand white rabbits as immune animals, the animals are immunized with the immune antigen, and preferably subjected to booster immunization once every 14 days, for a total of 4 times, and then carotid artery blood of the animals is taken to obtain the antiserum.

When other animals are selected as immune animals, the immune interval time and immune times can be adjusted according to specific conditions.

The antiserum can also be obtained by heart blood sampling and venous blood sampling.

In a second aspect, the present disclosure provides use of the antibody in preparing a kit for detecting blood brain barrier early injury of cerebral ischemic stroke.

Specifically, provided is a kit containing the antibody of the present disclosure. The kit can be used for detecting the blood brain barrier early injury of cerebral ischemic stroke.

The kit detects whether the blood brain barrier early injury of cerebral ischemic stroke occurs by means of the fact that the antibody of the present disclosure can specifically identify specific degradation fragments generated by degradation of the occludin protein caused by blood brain barrier early injury of cerebral ischemic stroke.

Preferably, the kit, which is an ELISA kit, includes a solid-phase carrier coated with the antibody of the present disclosure, a detection antibody, an enzyme-labeled antibody and a standard antigen.

The solid-phase carrier can be an ELISA plate, a polystyrene or polyvinyl chloride microreaction microplate, and a plastic pipe, etc.

Further, the use concentration of the antibody for coating the solid-phase carrier is 0.1-1 μg/ml.

More specifically, the present disclosure provides a method for preparing the solid-phase carrier:

(1) coating: diluting a specific antibody XW-OCLN-2 with a 200 mM NaHCO₃ buffer solution (pH 9.6) to a final concentration of 0.1-1 μg/ml, adding 100 μL of the diluted occludin-specific antibody solution to each well of an ELISA plate, and storing in a refrigerator of 4° C. overnight;

(2) rinsing: rinsing each well of the ELISA plate with PBST for 4 times, each time for 5-10 min;

(3) blocking: adding a blocking solution of 1-10% BSA, and incubating at 37° C. for 1-2 h; and

(4) rinsing again: rinsing for 4 times, and drying in the air.

A preparation method of 1000 mL of the PBST is: 2.72 g of Na₂HPO₄; 0.28 g of NaH₂PO₄; 9 g of NaCl; 1000 mL of double distilled water; and 500 μL of Tween 20.

The technical solution after the dosages of the above reagents or raw materials are expanded or reduced in equal proportion is substantially the same as the above preparation method. Therefore, the preparation method of the solid-phase carrier is not limited to the above specific dosage conditions.

For example, in the above preparation method, the NaHCO₃ buffer solution can be replaced by a Na₂CO₃/NaHCO₃ buffer solution (pH 9.6) with or without preservatives such as sodium azide.

The blocking solution can be replaced by different concentrations of gelatin, Casein, or serum of an animal such as goats and horses.

The detection antibody is an antibody of different species (Thermo Fisher, Product code 33-1500) that does not bind to the present specific antibody but can identify the occludin protein via detection.

The enzyme-labeled antibody is an enzyme-labeled secondary antibody from the same species as the detection antibody (Zsbio ZB-2305).

The standard antigen is an artificially-synthesized small peptide, which can bind to the antibody of the present disclosure.

In a third aspect, the present disclosure provides use of the antibody or kit in detecting the blood brain barrier early injury of cerebral ischemic stroke.

Specifically, a sample to be detected is detected by the kit of the present disclosure, so as to determine whether the sample to be detected is a patient with acute cerebral ischemic stroke that suffers from blood brain barrier early injury, according to a detection result (such as an optical density value).

Various embodiments of the present disclosure can have one or more of the following advantages.

The present disclosure provides a specific antibody which only specifically identities the degradation fragments of the occludin protein, but does not identify the full-length occludin protein, so that the influence of the full-length occludin protein in serum on the detection result can be eliminated, and the specificity and the accuracy of detecting the blood brain barrier early injury of cerebral ischemic stroke are obviously improved.

It should be understood that the foregoing general description and the following detailed description are merely exemplary and explanatory and are not intended to limit the present disclosure.

### BRIEF DESCRIPTION OF THE DRAWINGS

The accompanying drawings to be described herein are incorporated into this disclosure and constitute a part of this disclosure. These accompanying drawings show the embodiments of the present disclosure, and are used with this specification to explain principles of various embodiments of the present disclosure.

FIG. 1 shows the hydrophobic analysis of the sequence of a human occludin protein described in Example 1 of the present disclosure.

FIG. 2 is a western blot detection diagram of the occludin level in serum in Example 1 of the present disclosure.

FIG. 3 shows the level of specific degradation fragments of occludin in serum of a patient with acute cerebral ischemic stroke and in serum of a healthy person in Example 3 of the present disclosure.

### DETAILED DESCRIPTION

Various embodiments of the present disclosure will be further described in detail hereafter with reference to examples. It should be understood that the following examples are given for illustrative purposes only, and are not intended to limit the scope of the present disclosure. Those skilled in the art can make various modifications and replacements to the present disclosure, without departing from the purpose and spirit of the present disclosure.

Descriptions of some steps in the experimental methods used in the following examples may be skipped if they are known to those of ordinary skill in the art.

Descriptions of some materials, reagents, etc. used in the following examples may be skipped if they are commercially available and known to those of ordinary skill in the art.

Intravenous thrombolysis is currently recognized as one of the most effective methods to treat acute cerebral ischemic stroke. The intravenous thrombolysis can rescue the ischemic brain tissue to the greatest extent, improve the neurological function, reduce the disability degree of patients, and improve the quality of life. Tissue-type plasminogen activator (tPA) is currently the only thrombolytic drug approved by FDA for the treatment of acute cerebral ischemic stroke. However, the tPA thrombolysis has a very harsh time window limit. The safety period is within 3 hours of stroke occurrence, and a cautious period is in 3-4.5 hours.

Due to the harsh time window, the proportion of patients meeting thrombolytic therapy indications at the present stage is very low. Statistical figures show that, the proportion of patients who can reach the hospital within the time window (within 3-4.5 hours) and use the tPA for thrombolysis is very low: the proportion is less than 5% in developed countries, and less than 1.6% in China.

The main risk of delayed thrombolysis is increased complications of intracerebral hemorrhage, leading to condition aggravation or even death. Cerebral vascular damage is the main cause of the occurrence of hemorrhagic transformation. As early as before thrombolysis for cerebral ischemia, cerebral vessels had already been damaged, and with the extension of ischemia time, the vascular injury is aggravated. After blood flow recanalization upon thrombolytic therapy, the perfused blood leaks from the damaged blood vessels, resulting in hemorrhage. In addition, the activity of a blood fibrinolytic system is sthenic during the tPA thrombolysis, which makes it difficult to stop bleeding in time and aggravates bleeding once bleeding occurs.

Therefore, the inventors of the present disclosure have recognized that it is urgent to find a marker that can evaluate the degree of vascular injury in the early stage of onset, which can be used for screening those patients who are outside the thrombolytic time window (more than 4.5 h) but have good vascular conditions and can receive thrombolytic therapy, and those patients who are excluded from the thrombolytic time window of 3-4.5 h but have high risk of hemorrhagic transformation, thereby enabling more patients with cerebral stroke to receive thrombolytic therapy.

At present, a rapid and accurate method for detecting early vascular injury with a blood marker can help evaluate the risk of early hemorrhagic transformation, thereby guiding clinical tPA thrombolysis in early cerebral stroke. Moreover, the method can further predict the efficacy of a brain tissue protective drug, because most brain protective drugs cannot directly pass through the blood-brain barrier. Early blood brain barrier injury can also be used for evaluating brain injury and judging prognosis, so rapid detection of blood-brain barrier injury with the blood marker can also be used for evaluating brain injury degree of cerebral trauma.

Studies have found that, the degradation fragments of occludin can be used as blood markers to evaluate blood-brain barrier damage, thereby determining the degree of blood-brain barrier damage. However, there is currently no method for specifically identifying degradation fragments of occludin, so that the influence of the full-length occludin protein in serum on the detection result cannot be excluded. However, the identification of the full-length occludin protein in serum will affect the accuracy of the detection result and thus cause misjudgment.

Therefore, the inventors of the present disclosure have recognized that it is urgent to provide a substance or method that can specifically identify degradation fragments of occludin, so as to improve the specificity and accuracy of detection.

### Example 1: Development of Specific Antibody

This example was used for illustrating the development process of the specific antibody of the present disclosure. The specific steps were as follows:

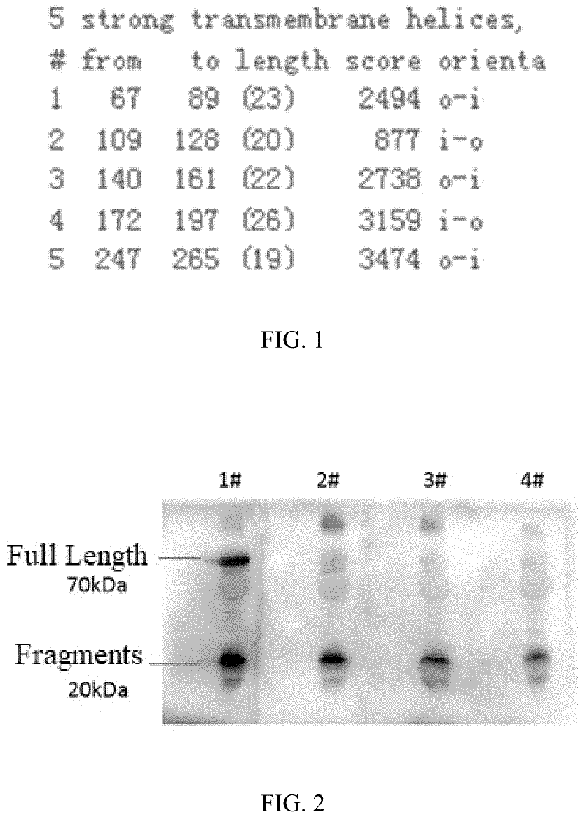

1. according to the sequence of the human full-length occludin protein (AAB00195.1, as shown in SEQ ID NO.1), through hydrophobic analysis (FIG. 1), it was determined that the protein was a multi-transmembrane protein, and the polypeptide sequence suitable for antigen recognition and antibody preparation was predicted. From N-terminal to C-terminal, the following 4 sequences were selected for polypeptide synthesis:

sequence 1#YRPDEFKPNHYAPSNC;
sequence 2#KTRRKMDRYDKSNIL;
sequence 3#DHYETDYTTGGESC;
sequence 4#SKLSHIKKMVGDYDR.

2. Synthesizing antigen:

the above-mentioned 1 #-4 # polypeptides were coupled with KLH respectively (with a coupling agent of SuLfo-SMCC) to obtain KLH-polypeptides as 1 #-4 # immune antigens;

the above-mentioned 1 #-4 # polypeptides were coupled with BSA respectively (with a coupling agent of glutaraldehyde) to obtain BSA-polypeptides as 1 #-4 # detection antigens.

3. New Zealand white rabbits were separately immunized with 1 #-4 # immune antigens by subcutaneous injection of the polypeptides. Booster immunization was conducted once every 14 days, for a total of 4 times. Carotid artery blood was taken to obtain antiserum.

4. An antigen affinity column was prepared by connecting the 4 polypeptides synthesized in the step 1 to an activated SuLfolink Resin.

By using the antigen affinity column, antibody purification of the antiserum obtained in the step 3 was conducted to obtain specific antibodies respectively, and the corresponding antibody numbers were XW-OCLN-1, XW-OCLN-2, XW-OCLN-3, and XW-OCLN-4. The collected eluate was tested for absorbance at 280 nm. Components with absorbance greater than 1.0 were combined and dialyzed against PBS. The dialyzed antibody was detected for protein concentration, and determined for antibody titer by ELISA.

5. Titers of the specific antibodies: immune response effects were detected by ELISA employing the BSA-polypeptides (1 #-4 # detection antigens) (Table 1).

TABLE 1
Antibody Titers detected by ELISA

| Antibody | Blank | Antibody Concentration | | | |
|---|---|---|---|---|---|
| No. | IgG | 1:80000 | 1:40000 | 1:20000 | 1:10000 |
| XW-OCLN-1 | 0.155512 | 4.05253 | 4.87158 | 4.96001 | 3.27384 |
| XW-OCLN-2 | 0.276284 | 5.22395 | 5.25199 | 5.28598 | 3.77305 |
| XW-OCLN-3 | 0.118125 | 1.3104 | 2.28969 | 4.71837 | 3.47192 |
| XW-OCLN-4 | 0.278177 | 4.66451 | 4.94387 | 5.06524 | 4.73881 |

The ELISA plates were respectively coated with 1 #-4 # detection antigens at a concentrations of 1 μg/ml, and specific antibodies (XW-OCLN-1, XW-OCLN-2, XW-OCLN-3, XW-OCLN-4) at different concentrations and blank IgG were accordingly added. The detection result showed that, the specific antibodies obtained from purification could specifically identify the detection antigen, and had a very high titer. When the concentration is 1:80000, a significant specific binding signal was still visible.

6. Detection of identification of the full-length occludin protein and fragments of occludin in the serum of a patient by antibodies.

The serum of the same patient with acute cerebral ischemic stroke was subjected to electrophoresis and membrane transfer, incubated with the antibodies XW-OCLN-1, XW-OCLN-2, XW-OCLN-3 and XW-OCLN-4 respectively, and detected for the level of occludin in the serum by western blot. The results were shown in FIG. 2.

As could be seen from FIG. 2, the XW-OCLN-1 antibody could identify the full-length occludin protein and protein fragments of 20 kDa.

While, the antibodies XW-OCLN-2, XW-OCLN-3 and XW-OCLN-4 only identified the protein fragments, rather than the full-length occludin protein.

Therefore, it was determined that the antibodies XW-OCLN-2, XW-OCLN-3 and XW-OCLN-4 could specifically identify degradation fragments of occludin in serum, could eliminate the influence of the full-length occludin protein in serum on the detection result, and significantly improved the specificity of the detection result.

### Example 2 Kit for Specifically Detecting Blood Brain Barrier Early Injury of Cerebral Ischemic Stroke

In this example, by taking the specific antibody XW-OCLN-3 obtained in the step 4 of Example 1 as an example, a kit for specifically detecting the blood brain barrier early injury of cerebral ischemic stroke was illustrated. The kit, which was an ELISA kit, included a solid-phase carrier coated with the specific antibody XW-OCLN-3, a detection antibody, an enzyme-labeled antibody, and a standard antigen.

wherein:

A method for preparing the solid-phase carrier specifically included the steps:

(1) coating: diluting a specific antibody XW-OCLN-3 with a 200 mM NaHCO₃ buffer solution to a final concentration of 1 μg/ml, adding 100 μL of the diluted occludin-specific antibody solution to each well of an ELISA plate, and storing in a refrigerator of 4° C. overnight;

(2) rinsing: rinsing each well of the ELISA plate with PBST for 4 times, each time for 5 min;

(3) blocking: adding a blocking solution of 10% BSA, and incubating at 37° C. for 2 h; and

(4) rinsing again: rinsing for 4 times, and drying in the air at room temperature.

The preparation method of 1000 mL of the PBST was: 2.72 g of Na₂HPO₄; 0.28 g of NaH₂PO₄; 9 g of NaCl; 1000 mL of double distilled water; and 500 μL of Tween 20.

The detection antibody is an antibody of different species (Thermo Fisher, Product code 33-1500) that does not bind to the present specific antibody but can identify the occludin protein via detection.

The enzyme-labeled antibody is an enzyme-labeled secondary antibody from the same species as the detection antibody (Zsbio ZB-2305).

The standard antigen is an artificially-synthesized small peptide, which can bind to the specific antibody of the present disclosure.

### Example 3 Use of Detection Kit

This example was used for explaining how to use the ELISA kit prepared in Example 2 to detect the level of specific degradation fragments of occludin in the serum of a patient with acute cerebral ischemic stroke, so as to measure the blood brain barrier early injury in a patient with cerebral stroke.

In this example, by employing the ELISA kit prepared in Example 2, the serums of 6 patients with acute cerebral ischemic stroke and the serums of 5 healthy persons were detected.

(1) the serums of the patients with acute cerebral ischemic stroke and the serums of healthy patients who undergo physical examination were taken; 100 μL of human serum was added, incubation was conducted at 37° C. for 2 hours, and rinsing was conducted;

(2) 100 μL of the detection antibody (Thermo Fisher, product code 33-1500) was added, incubation was conducted for 2 h, and rinsing was conducted for 4 times;

(3) 100 μL of secondary antibody (Zsbio ZB-2305) was added, and the mixture was allowed to maintain in a warm bath at 37° C. for 30 min, and rinsed; and

(4) 90 μL of a TMB substrate color developing solution was added into each well, color development was conducted in a dark place at 37° C., and 30 min later, the optical density value was detected at a wavelength of 450 nm by a microplate reader. According to the known optical density values of standard substances at different concentrations, a standard curve was plotted, and the level of the fragments of occludin in serum was obtained according to the standard curve.

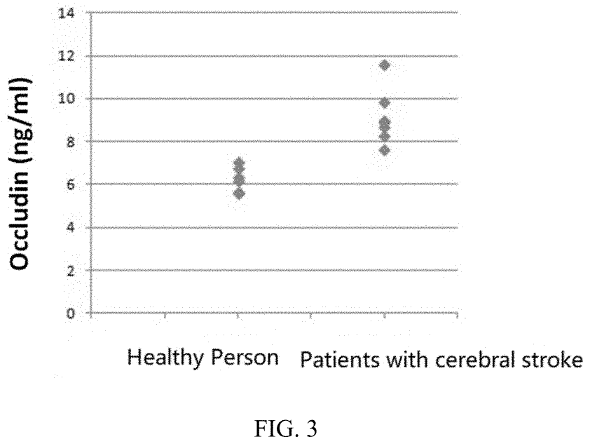

Results were as shown in FIG. 3. It could be seen from the FIG. 3 that, the level of specific degradation fragments of occludin in serum of patients with acute cerebral ischemic stroke was significantly higher than that of healthy patients who undergo physical examination, indicating that the kit could identify the specific degradation fragment of occludin in serum.

It should be understood that, the technical solution, in which the dosages of reagents or raw materials used in the above-mentioned examples are expanded or reduced in equal proportion, is substantially the same as the above-mentioned examples.

Although the present disclosure has been described in detail with general description and specific embodiments hereinabove, it is obvious to those skilled in the art that some modifications or improvements can be made on the basis of the present disclosure. Therefore, all such modifications or improvements made without departing from the spirit of the present disclosure are within the claimed scope of the present disclosure.

The antibody capable of specifically identifying degradation fragments of occludin in serum, as disclosed by the present disclosure, can be used for detecting a patient, can eliminate the influence of the full-length occludin protein in serum on the detection result, and obviously improves the specificity of the detection result. By detecting the concentration of protein fragments in a sample, it can be used for judging whether cerebral ischemic stroke occurs and the degree of onset.

While this specification contains many specific implementation details, these should not be construed as limitations on the scope of any claims, but rather as descriptions of features specific to particular implementations. Certain features that are described in this specification in the context of separate implementations can also be implemented in combination in a single implementation. Conversely, various features that are described in the context of a single implementation can also be implemented in multiple implementations separately or in any suitable subcombination.

Moreover, although features can be described above as acting in certain combinations and even initially claimed as such, one or more features from a claimed combination can in some cases be excised from the combination, and the claimed combination can be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the implementations described above should not be understood as requiring such separation in all implementations, and it should be understood that the described components and systems can generally be integrated together in a single product or packaged into multiple products.

As such, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking or parallel processing can be utilized.

It is intended that the specification and embodiments be considered as examples only. Other embodiments of the disclosure will be apparent to those skilled in the art in view of the specification and drawings of the present disclosure. That is, although specific embodiments have been described above in detail, the description is merely for purposes of illustration. It should be appreciated, therefore, that many aspects described above are not intended as required or essential elements unless explicitly stated otherwise.

Various modifications of, and equivalent acts corresponding to, the disclosed aspects of the example embodiments, in addition to those described above, can be made by a person of ordinary skill in the art, having the benefit of the present disclosure, without departing from the spirit and scope of the disclosure defined in the following claims, the scope of which is to be accorded the broadest interpretation so as to encompass such modifications and equivalent structures.

1. An antibody for specifically detecting blood brain barrier early injury of cerebral ischemic stroke, wherein the antibody only specifically identifies DHYETDYTTGGESC in degradation fragments of an occludin protein, but does not identify the occludin protein at full length.

2. The antibody according to claim 1, wherein the antibody is prepared with a method comprising:
(1) synthesizing a polypeptide DHYETDYTTGGESC;
(2) synthesizing an antigen: coupling the polypeptide synthesized in the step (1) with KLH to obtain KLH-polypeptide as an immune antigen;
(3) immunizing animals with the immune antigen to obtain antiserum; and
(4) performing antibody purification on the antiserum with an antigen affinity column to obtain a specific antibody.

3. The antibody according to claim 2, wherein the animals are New Zealand white rabbits.

4. The antibody according to claim 3, wherein the animals are immunized with the immune antigen, and subjected to booster immunization once every 14 days, for a total of 4 times, and then carotid artery blood of the animals is taken to obtain the antiserum.

5. The antibody according to claim 2, wherein the animals are immunized with the immune antigen, and subjected to booster immunization once every 14 days, for a total of 4 times, and then carotid artery blood of the animals is taken to obtain the antiserum.

6. A kit containing the antibody according to claim 1.

7. The kit according to claim 6, wherein the kit is an ELISA kit, and comprises a solid-phase carrier coated with the antibody, a detection antibody, an enzyme-labeled antibody and a standard antigen.

8. The kit according to claim 7, wherein the used concentration of the antibody coating the solid-phase carrier is 0.1-1 μg/ml.

9. The kit according to claim 7, wherein the standard antigen is an artificially-synthesized small peptide capable of binding to the antibody.

10. A kit containing the antibody according to claim 2.

11. Use of the antibody according to claim 1 in preparing a kit for detecting blood brain barrier early injury of cerebral ischemic stroke.

12. The use according to claim 11, wherein the kit is an ELISA kit, and comprises a solid-phase carrier coated with the antibody, a detection antibody, an enzyme-labeled antibody and a standard antigen.

13. Use of the kit according to claim 6 in detecting the blood brain barrier early injury of cerebral ischemic stroke.

14. A method of using the kit according to claim 6, wherein the antibody capable of specifically identifying degradation fragments of occludin in serum, the method comprising determining whether the ischemic stroke occurs and a degree of onset in a patient based on the antibody, eliminating influence of the full-length occludin protein in serum on a detection result, and improving specificity of the detection result.

15. The method of claim 14, further comprising detecting a concentration of protein fragments in a sample of the patient.

16. The method of claim 15, wherein the detecting comprises:
(1) obtaining the serum of the patient, adding 100 μl of human serum, incubating at 37° C. for 2 hours, and conducting rinsing;
(2) adding 100 μL of detection antibody, incubating for 2 hours, and performing rinsing for 4 times;
(3) adding 100 μL of secondary antibody, and maintaining the sample in a warm bath at 37° C. for 30 minutes, and performing rinsing; and
(4) adding 90 μl of a TMB substrate color developing solution into each well of sample, performing color development in a dark environment at 37° C., and detecting, after 30 minutes, an optical density value at a wavelength of 450 nm with a microplate reader, based on known optical density values of standard substances at different concentrations, plotting a standard curve was plotted, and obtaining a level of the fragments of occludin in the serum according to the standard curve;
the method further comprising determining the patient with acute cerebral ischemic stroke based on a significantly higher level of the specific degradation fragments of occludin than that of healthy subjects.

17. The method of claim 16, wherein the antibody is obtained by:
(1) according to a sequence of human full-length occludin protein (AAB00195.1), through hydrophobic analysis, determining that the protein was a multi-transmembrane protein, and predicting a polypeptide sequence suitable for antigen recognition and antibody preparation; selecting, from N-terminal to C-terminal, following 4 sequences for polypeptide synthesis:
sequence 1#YRPDEFKPNHYAPSNC;
sequence 2#KTRRKMDRYDKSNIL;
sequence 3#DHYETDYTTGGESC;
sequence 4#SKLSHIKKMVGDYDR;
(2) Synthesizing antigen:
coupling the sequences 1 #-4 # polypeptides with KLH respectively (with a coupling agent of SuLfo-SMCC) to obtain KLH-polypeptides as 1 #-4 # immune antigens;
coupling the sequences 1 #-4 # polypeptides with BSA respectively (with a coupling agent of glutaraldehyde) to obtain BSA-polypeptides as 1 #-4 # detection antigens;
(3) separately immunizing New Zealand white rabbits with 1 #-4 # immune antigens by subcutaneous injection of the polypeptides; conducing booster immunization once every 14 days, for a total of 4 times; taking carotid artery blood to obtain antiserum;
(4) preparing an antigen affinity column by connecting the 4 polypeptides synthesized in step (1) to an activated SuLfolink Resin;
by using the antigen affinity column, antibody purification of the antiserum obtained in the step (3) is conducted to obtain specific antibodies respectively, and corresponding antibody numbers are XW-OCLN-1, XW-OCLN-2, XW-OCLN-3, and XW-OCLN-4;
testing the collected eluate for absorbance at 280 nm; components with absorbance greater than 1.0 are combined and dialyzed against PBS; dialyzed antibody is detected for protein concentration, and determined for antibody titer by ELISA;
(5) titers of the specific antibodies: immune response effects are detected by ELISA employing the BSA-polypeptides (1 #-4 # detection antigens); and
(6) detecting identification of the full-length occludin protein and fragments of occludin in the serum by antibodies; wherein the serum of the patient with acute cerebral ischemic stroke is subjected to electrophoresis and membrane transfer, incubated with the antibodies XW-OCLN-1, XW-OCLN-2, XW-OCLN-3 and XW-OCLN-4 respectively, and detected for the level of occludin in the serum by western blot;

18. The method of claim 17, wherein the kid is an ELISA kit, and is included a solid-phase carrier coated with the specific antibody XW-OCLN-2, a detection antibody, an enzyme-labeled antibody, and a standard antigen.

19. The method of claim 18, wherein the solid-phase carrier is prepared by:
(a) coating: diluting a specific antibody XW-OCLN-2 with a 200 mM NaHCO₃ buffer solution to a final concentration of 1 μg/ml, adding 100 μL of the diluted occludin-specific antibody solution to each well of an ELISA plate, and storing in a refrigerator of 4° C. overnight;
(b) rinsing: rinsing each well of the ELISA plate with PBST for 4 times, each time for 5 min;
(c) blocking: adding a blocking solution of 10% BSA, and incubating at 37° C. for 2 h; and
(d) rinsing again: rinsing for 4 times, and drying in the air at room temperature.

20. The method of claim 19, further comprising preparing 1000 mL of the PBST including: 2.72 g of Na2HPO4; 0.28 g of NaH2PO4; 9 g of NaCl; 1000 mL of double distilled water; and 500 μl of Tween 20;
wherein the detection antibody is an antibody of different species (Thermo Fisher, Product code 33-1500) that does not bind to the specific antibody but is configured to identify the occludin protein via detection; and
wherein the enzyme-labeled antibody is an enzyme-labeled secondary antibody from the same species as the detection antibody (Zsbio ZB-2305).


SEQUENCE LISTING (1 sequence)

SEQ ID NO: 1 - protein, 522 aa, Homo sapiens
MSSRPLESPPPYRPDEFKPNHYAPSNDIYGGEMHVRPMLSQPAYSFYPEDEILHFYKWTSPPGVIRILSMLIIVMCIAIFACVASTLAWDRGYGTSLLGGSVGYPYGGSGFGSYGSGYGYGYGYGYGYGGYTDPRAAKGFMLAMAAFCFIAALVIFVTSVIRSEMSRTRRYYLSVIIVSAILGIMVFIATIVYIMGVNPTAQSSGSLYGSQIYALCNQFYTPAATGLYVDQYLYHYCVVDPQEAIAIVLGFMIIVAFALIIFFAVKTRRKMDRYDKSNILWDKEHIYDEQPPNVEEWVKNVSAGTQDVPSPPSDYVERVDSPMAYSSNGKVNDKRFYPESSYKSTPVPEVVQELPLTSPVDDFRQPRYSSGGNFETPSKRAPAKGRAGRSKRTEQDHYETDYTTGGESCDELEEDWIREYPPITSDQQRQLYKRNFDTGLQEYKSLQSELDEINKELSRLDKELDDYREESEEYMAAADEYNRLKQVKGSADYKSKKNHCKQLKSKLSHIKKMVGDYDRQKT